(19) United States
(12) Patent Application Publication
(10) Pub. No.: US 2020/0054775 A1
(43) Pub. Date: Feb. 20, 2020

(54) VALVED CHAMBER FOR LUMEN DEVICE AND METHODS OF USING THE SAME

(71) Applicant: Medivators Inc., Minneapolis, MN (US)

(72) Inventor: Michael P. Petersen, Eden Prairie, MN (US)

(21) Appl. No.: 16/487,498

(22) PCT Filed: Feb. 28, 2018
(86) PCT No.: PCT/US2018/020198

§ 371 Date: Aug. 21, 2019

Related U.S. Application Data
(60) Provisional application No. 62/468,189, filed on Mar. 7, 2017.

(51) Int. Cl. A61L 2/20; A61B 1/12; A61L 2/26; A61L 2/22

(52) U.S. Cl. CPC A61L 2/208; A61B 1/123; A61L 2/26; A61L 2/20; A61L 2/22; A61L 2202/24; A61L 2202/15; A61L 2202/123; A61L 2202/11; A61L 2202/14; A61L 2202/122

(57) ABSTRACT

Disclosed herein is a system for decontaminating a device having a lumen. The system includes a pressure vessel defining an enclosed space having a fixed volume, and the pressure vessel has an opening. The system includes a valve positioned at the opening of the pressure vessel which is controllable between an open first position and a closed second position. The system includes a container configured to receive a device having a lumen, the container defining a wall having a section permeable to vaporized decontaminating substance. The enclosed space of the pressure vessel is configured to be in fluid communication with the lumen of the device and the valve configured to control a flow between the lumen and the enclosed space.

### CROSS REFERENCE TO RELATED APPLICATIONS

This application claims priority to U.S. Provisional Patent Application Ser. No. 62/468,189 filed Mar. 7, 2017. This application is incorporated herein by reference, in its entirety.

### TECHNICAL FIELD

The present disclosure relates to decontamination of devices, such as medical devices. More particularly, the present disclosure relates to systems and methods for decontaminating medical devices having a lumen.

### BACKGROUND

Devices that include elongated and/or tortious flow paths often present certain challenges for decontamination. An example of a device that has an elongated flow path that may require repeated decontamination processing is an endoscope. An endoscope is an optic instrument that is used to inspect and treat interior portions of the body. Endoscopes have elongated lumens that are used to direct fluids, air, or tools into the body. Endoscopes may present certain problems in that such devices typically have numerous exterior crevices and interior lumens which can harbor microbes. Microbes can be found on surfaces in such crevices and interior lumens as well as on exterior surfaces of the endoscope. Other medical or dental instruments which comprise lumens, crevices, and the like can also provide challenges for decontaminating various internal and external surfaces that can harbor microbes.

Medical instruments formed of rubber or plastic components with adhesives are delicate and often unsuited to the high temperatures and pressures associated with processing in steam autoclaves. Steam autoclaves often operate under pressure cycling programs to increase the rate of steam penetration into the medical devices or associated packages of medical devices undergoing sterilization. Steam sterilization using gravity, high pressure, or pre-vacuum creates an environment where rapid changes in temperature or pressure can take place. Complex instruments which are often formed and assembled with very precise dimensions, close assembly tolerances, and sensitive optical components, such as endoscopes, may be destroyed or have their useful lives severely curtailed by harsh sterilization methods employing high temperatures and high or low pressures.

There is thus a need for a decontamination system or process that can be used to adequately decontaminate a device having a lumen without risking damage to the device.

### SUMMARY

Various aspects of the present disclosure relate to a system for decontaminating a device having a lumen. The system includes a pressure vessel defining an enclosed space having a fixed volume, and the pressure vessel has an opening. The system includes a valve positioned at the opening of the pressure vessel which is controllable between an open first position and a closed second position. The system includes a container configured to receive a device having a lumen, the container defining a wall having a section permeable to vaporized decontaminating substance. The enclosed space of the pressure vessel is configured to be in fluid communication with the lumen of the device and the valve configured to control a flow between the lumen and the enclosed space.

Various aspects of the present disclosure relate to a system for decontaminating a device having a lumen. The system comprises a decontamination chamber defining a first enclosed space. The system includes a container positioned within the first enclosed space for holding the device, and a source of decontaminating substance. The system includes at least one of a vaporizer or atomizer connected to the source of decontaminating substance and the decontamination chamber. The system includes a pressure vessel having an inlet and defining a second enclosed space. The pressure vessel is positioned within the first enclosed space. The system includes a valve connected to the inlet of the pressure vessel and configured to connect to the lumen to provide fluid communication between the second enclosed space and the lumen.

Various aspects of the present disclosure relate to a method of decontaminating a device having a lumen. The method includes enclosing a device having a lumen and a pressure vessel in a decontamination chamber. An inlet of the pressure vessel is connected to the device by a valve such that the lumen forms at least a portion of a fluid path between the decontamination chamber and the pressure vessel when the valve is in an open position and the decontamination chamber and the pressure vessel are not in fluid communication when the valve is in a closed position. The method includes decreasing the pressure within the decontamination chamber to a first pressure with the valve in the open position. The method includes, after decreasing the pressure within the decontamination chamber to a first pressure, providing a vaporized decontaminating substance to and increasing the pressure within the decontamination chamber to a second pressure with the valve in the closed position. The method includes opening the valve to create a turbulent flow of the vaporized decontaminating substance through the lumen.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of a decontamination system according to some embodiments.

FIG. 2 is a flow chart of an exemplary decontaminating cycle of a decontamination process.

FIG. 3 is a flow chart of an exemplary venting cycle of a decontamination process.

FIG. 4 is a schematic view of a decontamination system according to some embodiments.

FIG. 5 is a schematic view of a decontamination system according to some embodiments.

FIG. 6 is a schematic view of a decontamination system for a device having multiple lumens, according to some embodiments.

### DETAILED DESCRIPTION

Devices, such as medical devices, can be decontaminated or sterilized at relatively low temperatures using hydrogen peroxide (H₂O₂) and/or peracetic acid (PAA) chemistry. In such systems, the chemistry may be provided as a vapor into a decontamination chamber containing the device to be decontaminated. Devices containing a lumen may be particularly challenging to decontaminate as there must be flow of the decontaminating substance through the lumen to allow decontaminating substance to contact the surface of the lumen. The instant disclosure describes a decontamination system which provides a pressure vessel that may be used to provide a flow of air containing decontaminating substance through a lumen device to achieve decontamination of the lumen device. The instant disclosure describes a system which provides turbulent flow of air containing decontaminating substance through the lumen. A method of using is also described.

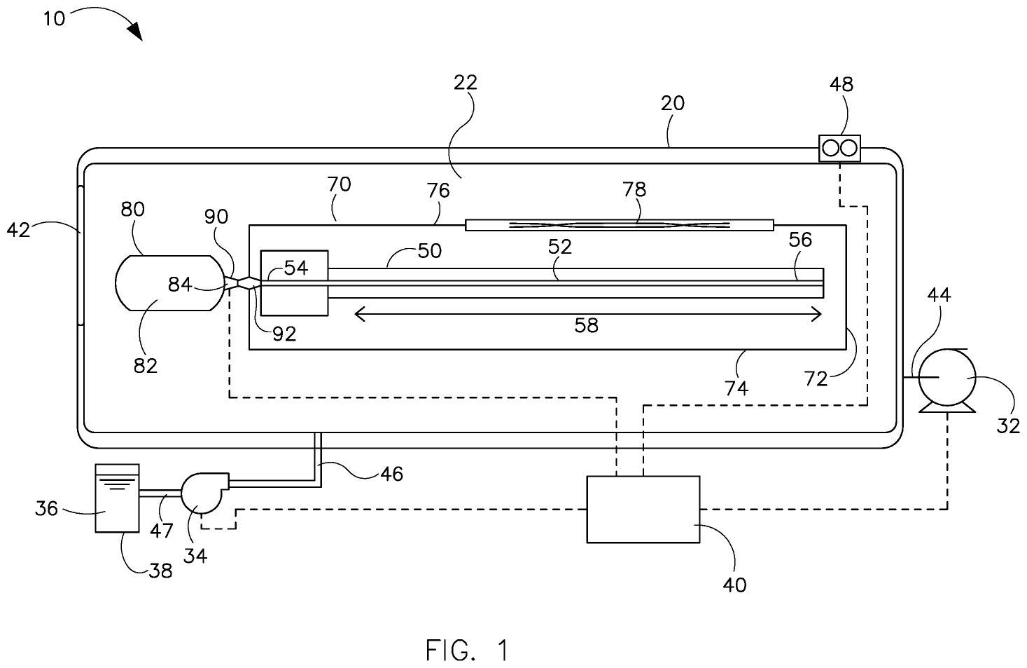

FIG. 1 is a schematic view of a decontamination system 10 which includes a decontamination chamber 20, a vacuum pump 32, a vaporizer 34, a source of decontaminating substance 36 maintained in a package 38, a controller 40, a vent 48, a device 50 positioned within a container 70, and a pressure vessel 80 including a valve 90. The vacuum pump 32 is connected to the decontamination chamber 20 by a conduit 44. The vaporizer 34 is connected to the decontamination chamber 20 by a conduit 46. The decontaminating substance 36 is maintained in the package 38 and is connected to the vaporizer 34 by a conduit 47. The controller 40 is connected to the vacuum pump 32, vaporizer 34, vent 48, pressure vessel 80 and/or valve 90 and is configured to control these items. As shown in FIG. 1, the decontamination system 10 contains a pressure vessel 80 within the decontamination chamber 20, although alternative configurations are also envisioned. This is described further below.

The decontamination chamber 20 defines an enclosed space 22. The decontamination chamber 20 includes a door 42 that is configured to accommodate placing items inside or removing items, such as the container 70 and the pressure vessel 80, from the decontamination chamber 20. The decontamination chamber 20 is configured to withstand pressure changes within the decontamination chamber 20. This is described further below. The door 42 may be sealed and/or reinforced to provide a sealed environment within the decontamination chamber 20 and maintains a pressure within the enclosed space 22 that is higher or lower than a pressure outside the decontamination chamber 20. The decontamination chamber 20 may be configured to receive the device 50 within the container 70.

The vacuum pump 32 is connected to the decontamination chamber 20 via the conduit 44 and is configured to change the pressure within the decontamination chamber 20. For example, the vacuum pump 32 may be configured to draw gas such as air from the decontamination chamber 20 to lower the pressure inside the decontamination chamber 20. The vacuum pump 32 may also be operated in the opposite manner such that the vacuum pump 32 forces gas into the decontamination chamber 20 to increase the pressure inside the decontamination chamber 20.

The decontamination chamber 20 also includes the vent 48, which may be placed in an open or closed position. When the vent 48 is in an open position, air can flow through the vent 48 and into or out of the decontamination chamber 20. For example, the vent 48 may be opened to allow the pressure inside the decontamination chamber 20 to equalize with the pressure outside the decontamination chamber 20. The position of vent 48 may be coordinated with operation of the vacuum pump 32. For example, the vent 48 may be closed when the vacuum pump 32 is lowering or raising the pressure inside the decontamination chamber 20. Additionally or alternatively, the vent 48 may be opened when the vacuum pump 32 is drawing air from within the decontamination chamber 20 to flush air from outside the decontamination chamber 20 through the decontamination chamber 20.

As shown in FIG. 1, the package 38 containing the decontaminating substance 36 and the vaporizer 34 are located outside the decontamination chamber 20. The package 38 is connected to the vaporizer 34 by conduit 47 and the vaporizer 34 is connected to the decontamination chamber 20 by conduit 46. Together, conduits 46, 47 provide a fluid connection from the package 38 into the decontamination chamber 20 such that the decontaminating substance 36 flows from the package 38 to the vaporizer 34 and into the decontamination chamber 20.

The decontaminating substance 36 may include chemistry suitable for use in a decontamination or sterilization process. For example, the decontaminating substance 36 may include peracetic acid (PAA) and/or hydrogen peroxide (H₂O₂). The decontaminating substance 36 may include a chemical or other substance that complies with the International Organization for Standardization (ISO) standard ISO/TC 198, Sterilization of Healthcare Products and/or the Association for the Advancement of Medical Instrumentation (AAMI) standard ANSI/AAMI/ISO 11140-1:2005, “Sterilization of Healthcare Products—Chemical Indicators—Part I: General Requirements” (Arlington, Va.: AAMI 2005). Decontaminating substance 36 may include chemistry that can be dispersed as a fluid, such as a liquid, a vapor, or a combination thereof (such as a fog) during a decontamination process. The decontaminating substance 36 may be kept at room temperature (e.g., 20° C. to 25° C.) before being provided to the vaporizer 34. In some embodiments, the decontaminating substance 36 may be cooled or heated above or below room temperature before being provided to the vaporizer 34.

The vaporizer 34 converts the decontaminating substance 36 into a vapor, fog, gas, or other suitable form for a decontamination process. For example, the vaporizer 34 may heat the decontaminating substance 36 provided in a liquid form to evaporate or otherwise transform the liquid decontaminating substance 36 into a vapor or gas. In an alternative configuration, the vaporizer 34 may convert the decontaminating substance 36 into a vapor or fog via a mechanical means such as an atomizing nozzle or a sprayer (e.g. the vaporizer may include an atomizer that uses a mechanical force such as rotating blades or air pressure to break up a stream of liquid decontaminating substance 36 into individual droplets and/or to produce an aerosol). The droplets or aerosol of decontaminating substance 36 may be released into the decontamination chamber 20 where they form a vapor. The vaporizer 34 may be controlled to fill the decontamination chamber 20 with air containing vaporized decontaminating substance 36 at a suitable temperature, pressure, relative humidity, and/or concentration of decontaminating substance 36. In some embodiments, the decontaminating substance 36 may be pulled into the vaporizer 34. In other embodiments, the decontaminating substance 36 may be pushed into the vaporizer 34.

The controller 40 provides control signals to and/or receives condition sensing and equipment status signals from other elements of the decontamination system 10. For example, the controller 40 may include monitoring and control of the vaporizer 34, the vacuum pump 32, the vent 48, the valve 90, and the pressure vessel 80. The controller 40 may regulate delivery of the decontaminating substance 36 to the vaporizer 34. The controller 40 may be configured to adjust the environmental conditions within the decontamination chamber 20 by controlling the vacuum pump 32, the vent 48, and/or the vaporizer 34. For example, the controller 40 may control the vacuum pump 32 for adjustment of the pressure of the decontamination chamber 20. The controller 40 may control the vaporizer 34 for adjustment of the relative humidity, the temperature, and/or the concentration of decontaminating substance 36 in the air within the decontamination chamber 20.

The container 70 forms an enclosed space and holds at least one device 50. The container 70 may have one or more sides 72 that form the enclosed space. Sides 72 may be flexible or rigid. The sides 72 of container 70 may be of the same material or different materials. The container 70 may be a flexible pouch made entirely or substantially entirely from one or more pliable or flexible material. The container 70 may be a case or other enclosure formed from a rigid material. In a further example, the container 70 may have a rigid bottom 74 and sides 72 and may have a flexible top 76 or lid. For example, the sides 72 and/or bottom 74 and top 76 may include walls that define an enclosed space and are configured to form a barrier around the device 50 to prevent the device 50 from contacting contaminating surfaces. In some embodiments, the container 70 may be disposable. In other embodiments, the container 70 may be reusable. The container 70 may be designed to contain the device 50 during a decontamination process, and maintain the device 50 in a decontaminated condition after the device 50 is removed from the decontamination chamber 20. The container 70 may be configured to enclose the device 50 and prevent contamination of the device 50 when the container 70 is removed from the decontamination chamber 20 and until the container 70 is opened.

The container 70 may have a section 78 on the sides 72 or the top 76 through which a gas such as air containing vaporized decontaminating substance 36 may pass. For example, the sides 72 or top 76 may include a section 78 that includes a material that allows air to pass through, but inhibits contaminating material and/or microbes from contacting the device 50. Vaporized decontaminating substance 36 in the decontamination chamber 20 may contact the sides 72, top 76, and bottom 74 of the container 70. Air containing vaporized decontaminating substance 36 may pass through the section 78 into or out of the container 70. In this manner, vaporized decontaminating substance 36 in the decontamination chamber 20 may pass through the section 78, and enter the container 70. Suitable materials that may be used to form the section 78 include, for example, a nonwoven material such as that sold under the tradename Tyvek®.

The device 50 may include a lumen 52 having a first end 54, a second end 56, and a length 58. The lumen 52 extends the length 58 of the device 50, and has an inner diameter that is narrow in comparison to the length 58 of the lumen 52. For example, the device 50 may be a medical device, such as an endoscope. An endoscope may have a rigid or flexible lumen 52 that extends the length of the endoscope. An endoscope lumen 52 may be as short as about 0.5 meters, 1.0 meter, or about 1.5 meters, in length or as long as about 2.0 meters, 3.0 meters, or 4.0 meters in length, or a length between any pair of the foregoing values. An endoscope lumen 52 may have an inner diameter as small as about 0.5 mm, 1.0 mm, or about 1.5 mm, or as wide as about 2.0 mm, 3.0, or about 3.5 mm, or a diameter between any pair of the foregoing values. An endoscope may have an outer diameter of as small as about 2.0 mm, 3.0 mm, or about 4.0 mm, or as wide as about 8.0 mm, 9.0 mm, or about 9.5 mm, or a diameter between any pair of the foregoing values. In some embodiments, the device 50 may include more than one lumen 52.

As shown in FIG. 1, the pressure vessel 80 is positioned within the decontamination chamber 20 outside the container 70 and is connected to the lumen 52 through the valve 90 and port 92. The inside of the pressure vessel 80 defines an enclosed space 82 that has a fixed volume. The enclosed space 82 may have a volume of as small as about 0.01 liters, 0.1 liters, 0.5 liters, or about 1.0 liter, or as large as about 3.0 liters, 5.0 liters, or about 10.0 liters, or a volume between any pair of the foregoing values. The pressure vessel 80 has a single opening 84 that provides fluid communication into the enclosed space 82. The opening 84 defines both an inlet and an outlet between the enclosed space 82 and the outside of the pressure vessel 80. In some embodiments, the fixed volume of the enclosed space 82 is greater than an internal volume of the lumen 52 (e.g. as small as about two times, about three times, or about five times the size of the internal volume of the lumen 52, or as large as about ten times, fifteen times, or twenty times the size of the internal volume of the lumen, or a volume between any pair of the foregoing values, for example, although other sizes are contemplated). The pressure vessel 80 is configured to withstand pressure changes, such as from atmospheric pressure to sub-atmospheric pressure inside the pressure vessel 80, without changing shape or volume. For example, during a decontamination cycle, a vacuum can be drawn inside the enclosed space 82 without the pressure vessel 80 changing volume. The pressure vessel 80 is formed of a rigid material that can withstand pressure changes and maintain structural integrity. Materials that the pressure vessel 80 may be formed from include metal, plastic, glass, or a composite material such as fiber glass. Suitable materials for forming the pressure vessel 80 also include materials sold under the tradenames of Plexiglas® or Kevlar®.

As shown in FIG. 1, the valve 90 is positioned at the opening 84 of the pressure vessel 80 and is configured to regulate flow into and out of the enclosed space 82 of the pressure vessel 80. For example, when the valve 90 is in a closed position, the pressure vessel 80 is completely enclosed and flow of a gas such as air into and out of the pressure vessel 80 is inhibited, such that the pressure within the pressure vessel 80 is maintained. When the valve 90 is in an open position, flow of a gas such as air into and out of the pressure vessel 80 is allowed. With the valve in the open position, gas such as air can flow between the pressure vessel 80 and the decontamination chamber 20 and the pressure within the pressure vessel 80 and the decontamination chamber 20 can equalize. The valve 90 is configured to withstand elevated pressures and vacuum conditions that may be provided within the decontamination chamber 20, for example during a decontamination process. The valve 90 may be sized and/or shaped to allow air and/or fluid to flow into or out of the pressure vessel 80 at a suitable flow rate.

The valve 90 may be configured to open at a suitable speed. That is, the valve 90 is configured to transition between the fully closed position and the open position at a suitable speed to allow air to flow into or out of the pressure vessel 80 at a suitable volumetric flow rate. The valve 90 may be a solenoid valve, which is an electromechanically operated valve that is controlled by an electric current through a solenoid and is suitable for rapidly transitioning between an open and closed position. For example, the valve 90 may be a direct-acting solenoid valve (e.g. having a power supply that directly controls a stopper across an opening) that is controllable to transition between an open and closed position in less than one second, for example, as low as about 5, 10, or 15 milliseconds, to as high as about 20, 30, or 50 milliseconds, or a value between any pair of the foregoing values. In some embodiments, the valve 90 is opened or closed at a speed configured to allow the pressure vessel 80 to fill or empty at a suitable rate.

As shown in FIG. 1, the valve 90 may be controlled with the controller 40 from outside the decontamination chamber 20. The valve 90 may be controlled by the controller 40 via a wired connection, that is, the valve 90 may have a direct connection to the controller 40 through a wire. Alternatively, the valve 90 may have a wireless connection with the controller 40. With the valve 90 controllable with a wireless connection, the valve 90 may be operated remotely. In alternative embodiments, the valve 90 may be configured to open or close independent of the controller 40. For example, the valve 90 may be configured to open or close at specific times or when a certain pressure is reached within the decontamination chamber 20. For instance, the valve 90 may be connected to a sensor positioned within the decontamination chamber 20, and the sensor may be configured to control the valve 90 between an open position and a closed position when a predetermined pressure is reached within the decontamination chamber 20 and/or the pressure vessel 80.

As shown in FIG. 1, the port 92 is positioned on the container 70 and connected to the device 50. The port 92 may be located on one of the sides 72, the top 76, or the bottom 74 of the container 70. The port 92 may be directly connected to the lumen 52. That is the port 92 may be connected to the lumen 52 without a space or gap between the port 92 and the lumen 52. As shown in FIG. 1, the port 92 is connected to the first end 54 of the lumen 52 and the valve 90. The port 92 may be attached to the valve 90 and/or the lumen 52 using any suitable connection such as a threaded connection that attaches or detaches by rotating the connection, or a quick snap connection that may be connected or disconnected by retracting or advancing a portion of the connection. The port 92 may include a layer of permeable material across the cross section of the port 92 that allows gas such as air containing vaporized decontaminating substance 36 to pass through and prevents contaminating substances and microbes from entering the container 70 through the port 92. The layer of permeable material across the port 92 prevents contamination of the lumen 52 after the container 70 is removed from the decontamination chamber 20. Suitable permeable materials include, for example, a nonwoven material such as that sold under the tradename Tyvek®.

In some embodiments, the lumen 52 is attached to the port 92, and the port 92 is attached to the valve 90 which is attached to the opening 84 of the pressure vessel 80. In this configuration, a flow path through the port 92, the valve 90 and the opening 84 defines a channel that allows fluid communication between the enclosed space 82 and the lumen 52. In some embodiments, the second end 56 of the lumen 52 may be open and flow of a gas may be allowed into the lumen 52 from the inside of the container 70, such that the lumen 52 is in fluid communication with the inside of the container 70. Gases such as air and/or vaporized decontaminating substance 36 within the enclosed space 22 of the decontamination chamber 20 may pass through the section 78 of the container 70, and into the container 70. When the valve 90 is in the open configuration, a gas such as air may flow between the inside of the container 70, the lumen 52, and the enclosed space 82 of the pressure vessel 80.

The valve 90 may be used to regulate a flow of gas such as air through the lumen 52. For example, with the lumen 52 directly connected to the port 92, and the port 92 directly connected to the valve 90, a flow of air through the lumen 52 may be controlled by opening or closing the valve 90. If the valve 90 is in the closed position, air is prevented from passing through the port 92 and a flow of air through the lumen 52 is prevented. If the port 92 is connected to the lumen 52 and the valve 90 is in the open position, a pressure difference between the inside of the pressure vessel 80 and the inside of the decontamination chamber 20 may provide a flow of air through the lumen 52.

In some embodiments, a pressure difference is formed between the inside of the pressure vessel 80 and the inside of the decontamination chamber 20 that is outside the pressure vessel 80 by controlling the valve 90 and the vacuum pump 32. For example, the vacuum pump 32 may be used to draw air from inside the decontamination chamber 20 to reduce the pressure inside the decontamination chamber 20 from a higher pressure to a lower pressure, such as a subatmospheric pressure (i.e., pump down). If the valve 90 is in the open configuration, air within the pressure vessel 80 will flow out of the pressure vessel 80, and the pressure within the pressure vessel 80 will be the same as the pressure outside the pressure vessel 80. If the valve 90 is in the closed configuration during the pump down, air within the pressure vessel 80 is inhibited from flowing out of the pressure vessel 80 and the pressure inside the pressure vessel 80 will be higher than the pressure outside the pressure vessel 80. Alternatively, the vacuum pump 32 may be used to pump air into the decontamination chamber 20 to increase the pressure inside the decontamination chamber 20 from a first (lower) pressure to a second (higher) pressure. If the valve 90 is in the open configuration during the pressure increase, the pressure inside the pressure vessel 80 will increase and will be the same as the air outside the decontamination chamber 20. If the valve 90 is in the closed configuration while the pressure outside the pressure vessel 80 increases, the pressure inside the pressure vessel 80 will remain at the first (lower) pressure, while the pressure outside the pressure vessel 80 increases to the second (higher) pressure. The decontamination system 10 may be configured to create a difference between the pressure inside of the pressure vessel 80 and a pressure outside of the pressure vessel 80 as low as about 5 Torr, about 20 Torr, about 50 Torr, or about 75 Torr, to as high as about 100 Torr, about 300 Torr, or about 760 Torr or a pressure difference between any pair of the foregoing values.

The valve 90 and vacuum pump 32 may be used in combination to provide a flow into and out of the pressure vessel 80. For example, the vacuum pump 32 may be used to pump air into or out of the decontamination chamber 20 to increase or decrease the pressure inside the enclosed space 22 of the decontamination chamber 20. As described above, if the valve 90 is in the closed position when the pressure inside the decontamination chamber 20 that is outside the pressure vessel 80 is changed, the pressure inside the pressure vessel 80 will be held constant while the pressure outside the pressure vessel 80 changes. Alternatively, if the valve 90 is in the open position while the pressure inside the pressure vessel 80 is different than the pressure outside the pressure vessel 80, gas such as air will flow through the valve 90 and the pressure inside the pressure vessel 80 will equalize with the pressure outside the pressure vessel 80. Whether the air inside the pressure vessel 80 is higher or lower than the pressure outside the pressure vessel 80 before the valve 90 is opened will determine whether the air flows into or out of the pressure vessel 80. Thus, using the vacuum pump 32 to change the pressure inside the decontamination chamber 20, in combination with controlling the valve 90 between the open and closed positions, flow into and out of the pressure vessel 80 can be provided. With the pressure vessel 80 connected to the port 92 through the valve 90, and the port 92 directly connected to the lumen 52, flow of air through the lumen 52 can also be provided.

In some embodiments, the decontamination system 10 may be used to provide air containing decontaminating substance 36 from the inside of the decontamination chamber 20 through the lumen 52. That is, the decontamination system 10 may be used to force a flow of air containing decontaminating substance 36 from the inside of the decontamination chamber 20 along the entire length 58 of the lumen 52. To provide a flow of air containing decontaminating substance 36 along the entire length 58 of the lumen 52, a volume of air at least as large as the volume of the lumen 52 is provided into the lumen 52 and allowed to travel the length 58 of the lumen 52. In this manner, the entire surface of the lumen 52 comes in contact with air containing decontaminating substance 36. The decontamination system 10 having the pressure vessel 80 may provide a volume of air containing decontaminating substance as small as about 0.5 liters, 1.0 liters, 2.0 liters, or about 3.0 liters, or as high as about 8.0 liters, 10.0 liters, or about 15.0 liters through each lumen 52.

The decontamination system 10 may be used to provide a suitable total amount of decontaminating substance 36 along the entire length 58 of the lumen 52 to achieve a desired level of decontamination within a suitable amount of time. For example, the concentration of vaporized decontaminating substance 36 in the air in the decontamination chamber 20 may be determined, and the required volume of air that contains a suitable amount of decontaminating substance 36 to decontaminate the entire lumen 52 may be forced or drawn through the lumen 52. In this manner, the entire surface of the lumen 52 can be contacted with decontaminating substance 36 to achieve a required level of decontamination in a suitable amount of time.

In some embodiments, the valve 90 may be sized such that in an open position, the valve 90 defines an opening wide enough to allow a suitable volumetric flow rate of air into the pressure vessel 80. The valve 90 may be sized such that in the fully open position, a flow path between the decontamination chamber 20 and the pressure vessel 80 has a diameter wide enough to allow a volumetric flow of air into the pressure vessel 80 that creates a turbulent flow of air through the lumen 52. In some embodiments, the valve 90 is opened at a suitable rate to allow a volumetric flow rate of air that creates turbulent flow through the lumen 52. Turbulent flow of air through the lumen 52 may be desired, for example, when providing air containing vaporized decontaminating substance 36 into the lumen 52. A turbulent flow of air containing decontaminating substance 36 inside the lumen 52 may provide suitable contact of the decontaminating substance 36 with the entire surface of the lumen 52.

In general, flow of compressible and/or incompressible fluids in a conduit may be laminar or turbulent. One method of modeling flow within a conduit as laminar or turbulent is by determining the Reynolds number for a given fluid at certain flow rates. The Reynolds number is a measure of the ratio of the inertial forces to the viscous forces of a fluid flow. A low Reynolds number correlates to laminar flow, and a high Reynolds number correlates to turbulent flow. (Perry, Robert H., Perry's Chemical Engineers' Handbook, 7^{th} Ed., Section 10-5, McGraw-Hill, (1997)). The Reynolds number is dimensionless, and for flow in a pipe it is calculated using Equation 1.

$$Re = \frac{ρ VD}{μ} \qquad Equation 1$$

Where ρ is the density of the fluid (e.g. kg/m³), V is the velocity of the fluid flow (e.g. m/s), D is the diameter of the pipe (e.g. m), and μ is the viscosity of the fluid (e.g. kg/ms). For most systems, a Reynolds number over 4000 is consistent with turbulent flow. The velocity of the fluid flow may be controlled by the volume of the enclosed space 82 inside the pressure vessel 80, the difference between the pressure inside the pressure vessel 80 and the pressure inside the decontamination chamber 20 outside the pressure vessel 80, and the diameter of the flow path.

In certain configurations, the pressure vessel 80 is sized with an internal volume large enough to produce a flow rate that produces turbulent flow through the lumen 52. For example, the volume of the enclosed space 82 is sized to provide a volumetric flow rate along the length 58 of the lumen 52 that provides a turbulent flow for lumens having certain diameters. In some embodiments, the volume of the pressure vessel 80 may be controllably adjustable and the volume of the pressure vessel 80 may be controlled to a suitable size for a given lumen, for example, based on the diameter and length of the lumen 52. Turbulent flow may also be created through additional parameters such as a shape or direction of a flow path. In some instances, a device such as an endoscope has a lumen 52 defining a flow path having rapid changes in directions, such as a bend, a curve, an elbow or junction that defines a flow path through an angle as low as about 5 degrees, 20 degrees, or about 30 degrees, to as high as about 90 degrees, 120 degrees, or about 179 degrees, or between any pair of the foregoing values. In a lumen 52 having one or more rapid changes in direction, turbulent flow may be formed by these changes in the direction of the flow path.

In certain applications, it can be challenging to achieve adequate decontamination of devices containing lumens, such as endoscopes, particularly long, narrow lumens of endoscopes. Material transfer along the length 58 of the lumen 52 of the endoscope is often difficult to achieve with passive diffusion of the chemistry. Generally, systems or methods using passive diffusion of chemistry along the entire length of a lumen 52 require long contact times and/or high chemistry concentrations to achieve adequate decontamination of the entire lumen 52. Long processing times and/or high concentrations increase the risk of damaging the endoscope. Extended exposure to high temperatures or high chemistry concentrations may damage certain materials used in an endoscope. Longer processing times generally also lead to higher operating costs, as fewer devices can be decontaminated in a given amount of time.

The aforementioned decontamination system 10 having a pressure vessel 80 provides faster decontamination of the lumen 52 and to a suitable decontamination level than an alternative system without a pressure vessel 80. A faster decontamination process allows a shorter amount of time required for each decontamination process, which increases the number of devices that can be processed with a single decontamination system 10 in a given amount of time. A faster decontamination process also reduces the amount of time the lumen 52 must be exposed to decontaminating substance 36 to achieve a suitable decontamination level. The decontamination system 10 described herein having a pressure vessel 80 enables a user to decontaminate a lumen 52 using a lower concentration of chemistry. The pressure vessel 80 allows a user to avoid potential damage to the device 50 because of the shorter exposure time and/or the lower concentration of chemistry required to achieve sufficient decontamination. Additionally, a lower concentration of the decontaminating substance 36 provides a decontaminating process that requires less decontaminating substance 36 and less time to operate the decontamination system 10, thus decreasing the costs of the decontamination process.

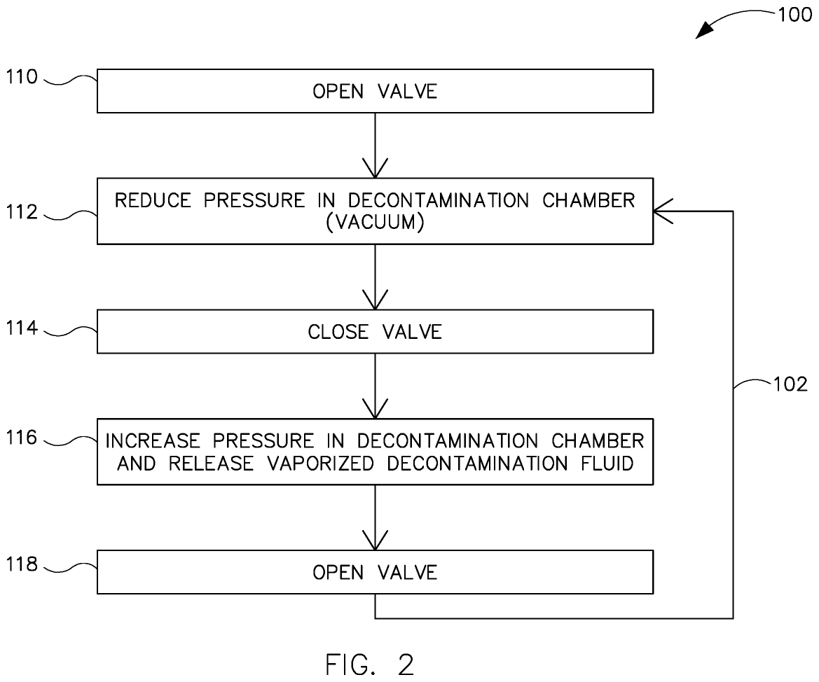

An example decontaminating cycle 100 for decontaminating a lumen using the decontamination system 10 in FIG. 1 is shown as a flow chart in FIG. 2. A decontaminating process may include the decontaminating cycle 100 repeated any suitable number of times as shown by arrow 102. In some embodiments, a decontaminating process begins when a device to be decontaminated is placed within the decontamination chamber. The device to be decontaminated includes a lumen. The device may be placed in a container. The lumen may be connected to the pressure chamber by connecting the lumen to a port that is connected to a valved opening on a pressure vessel. Alternatively, the lumen may be connected directly to the valved opening. With the lumen connected to the valved opening, the lumen forms a portion of a fluid path between the inside of the pressure vessel and the outside of the pressure vessel. After the device to be decontaminated has been connected to the valved opening, the decontamination chamber may be sealed, for example, by closing a door to the decontamination chamber. After the decontamination chamber is sealed, decontamination of the lumen may begin.

As shown in FIG. 2, the decontaminating cycle 100 begins with the valve in an open position in step 110. The valve may be in the open position before the decontamination chamber is sealed, or may be controlled in the open position after the decontamination chamber is sealed. After step 110, the pressure in the decontamination chamber is reduced in step 112, for example by removing air from the decontamination chamber with a vacuum pump. Step 112 may be continued until the pressure inside the decontamination chamber reaches a suitable level. For example, the pressure inside the decontamination chamber may be reduced in step 112 until the pressure reaches a suitable first pressure that is as low as about 0.1 Torr, about 1 Torr, about 5 Torr, or about 10 Torr, to as high as about 30 Torr, about 50 Torr, or about 100 Torr, or a pressure between any pair of the foregoing values, for example, although additional values are contemplated. In step 112, the pressure inside the pressure vessel may be monitored and the vacuum pump may be controlled to reduce the pressure inside the decontamination chamber until the pressure inside the pressure vessel is at a suitable pressure, such as a pressure as low as about 0.1 Torr, about 1 Torr, about 5 Torr, or about 10 Torr, to as high as about 30 Torr, about 50 Torr, or about 100 Torr, or between any pair of the foregoing values, for example, although additional values are contemplated. The vacuum pump is controlled to reduce the pressure within the decontamination chamber to a suitable level and the pressure inside the pressure vessel is allowed to equalize with the pressure inside the decontamination chamber. That is, the pressure inside the pressure vessel may be the same as the pressure inside the decontamination chamber.

In step 114, the valve is closed. In step 116, the pressure inside the decontamination chamber is increased. The pressure inside the decontamination chamber is increased to a second (higher) pressure by controlling the vaporizer and/or the vacuum pump to provide air and/or vaporized decontaminating substance to the inside of the decontamination chamber. The vaporizer is controlled to release vaporized or atomized decontaminating substance into the decontamination chamber which may raise the pressure inside the decontamination chamber. The vacuum pump may be controlled to provide air into the decontamination chamber to further increase the pressure in step 116.

After the pressure inside the decontamination chamber is increased to the second (higher) pressure in step 116, the valve is opened in step 118. Opening the valve allows air containing vaporized decontaminating substance to flow from the decontamination chamber into the pressure vessel through the valved opening. The valve may be opened with the difference between the pressure inside of the pressure vessel and the pressure outside of the pressure vessel as low as about 5 Torr, about 20 Torr, about 50 Torr, or about 75 Torr, to as high as about 100 Torr, about 300 Torr, or about 760 Torr, or a pressure difference between any pair of the foregoing values, for example, although additional values are contemplated. With the lumen forming a portion of a fluid path between the decontamination chamber and the valved opening, when the valve is opened, air from the decontamination chamber that is outside the pressure vessel flows through the lumen and into the pressure vessel. The volume of the pressure vessel determines the amount or volume of air that flows through the lumen. A suitable volume of air containing vaporized decontaminating substance is provided through the lumen and the decontaminating substance contacts the surfaces of the lumen. In some embodiments, a turbulent flow of air containing vaporized decontaminating substance is provided through the lumen.

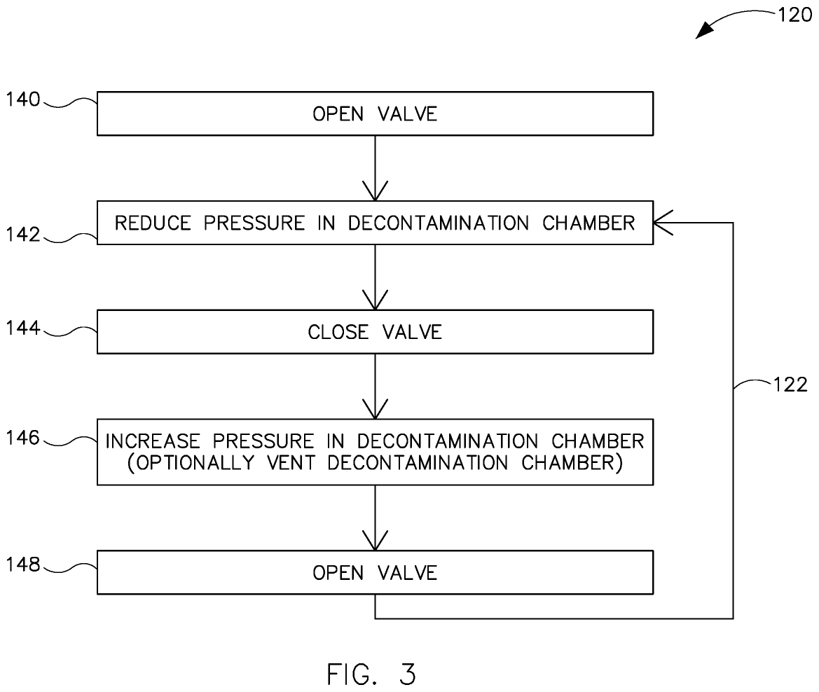

The vaporized decontaminating substance may be allowed to contact the surfaces of the lumen for a suitable period of time. In some instances, certain steps of decontaminating cycle 100 are repeated a suitable number of times as shown by arrow 102. After decontaminating cycle 100 is complete, decontaminating substance may be removed from the device, container, and decontamination chamber, for example with a venting cycle. An exemplary venting cycle 120 for removing decontaminating substance from the inside of the lumen is shown in the flow chart of FIG. 3. A decontaminating process may include the venting cycle 120 repeated any suitable number of times, as shown by arrow 122. For example, the venting cycle 120 may be repeated until a concentration of decontaminating substance in air that is exiting the decontamination chamber and/or inside the pressure vessel is below a suitable level. Venting cycle 120 of FIG. 3 may be carried out with system 10 of FIG. 1.

As shown in FIG. 3, venting cycle 120 begins with the valve controlled in an open position in step 140. For example, the valve may be open after the decontaminating cycle 100 shown in FIG. 2 is completed. With the valve in the open position in step 140, the pressure in the decontamination chamber is reduced in step 142, for example by removing air from the decontamination chamber with the vacuum pump. Step 142 may continue until the pressure inside the decontamination chamber reaches a predetermined level. For example, the pressure inside the decontamination chamber may be reduced in step 142 until the pressure reaches a suitable first (lower) pressure as low as about 0.1 Torr, about 1 Torr, about 5 Torr, or about 10 Torr, or as high as about 30 Torr, about 50 Torr, or about 100 Torr, or between any pair of the foregoing values. In step 142, the vacuum pump may be controlled to reduce the pressure inside the decontamination chamber until the pressure inside the pressure vessel is as low as about 0.1, about 1 Torr, about 5 Torr, or about 10 Torr, or as high as about 30 Torr, about 50 Torr, or about 100 Torr, or between any pair of the foregoing values. In some embodiments, the vacuum pump may reduce the pressure within the decontamination chamber to below a suitable level after which the pressure inside the pressure vessel is allowed to equalize with the pressure inside the decontamination chamber that is outside the pressure vessel.

After the pressure inside the pressure vessel reaches a suitable level, in step 144, the valve is closed. After the valve is closed the pressure inside the decontamination chamber may be increased in step 146. For example, the pressure inside the decontamination chamber may be increased to a second (higher) pressure by controlling the vent and/or the vacuum pump to provide air inside the decontamination chamber. For example, the vent may be opened to allow air to flow into the decontamination chamber and allow a pressure inside the decontamination chamber to reach atmospheric pressure. In some embodiments, the vacuum pump may add air into the decontamination chamber to further increase the pressure in step 146, for example, to a pressure that is greater than atmospheric pressure.

After the pressure inside the decontamination chamber is increased to the second (higher) level in step 146, the valve may be controlled to the open position in step 148. Opening the valve allows air free of decontaminating substance to flow from outside the pressure vessel into the pressure vessel. With the lumen connected to the pressure vessel, the lumen forms a portion of a fluid path between the inside of the pressure vessel and the outside of the pressure vessel. Air from the decontamination chamber that is outside the pressure vessel may flow through the lumen, through the pressure vessel opening, and into the pressure vessel. A suitable volume of air is allowed to flow through the lumen, allowing the lumen to vent with the air flow. The speed of the air flowing through the lumen may be controlled to form turbulent flow inside the lumen. Venting cycle 120 may be repeated any suitable number of times to ensure the amount of decontaminating substance remaining in the lumen is below a suitable amount.

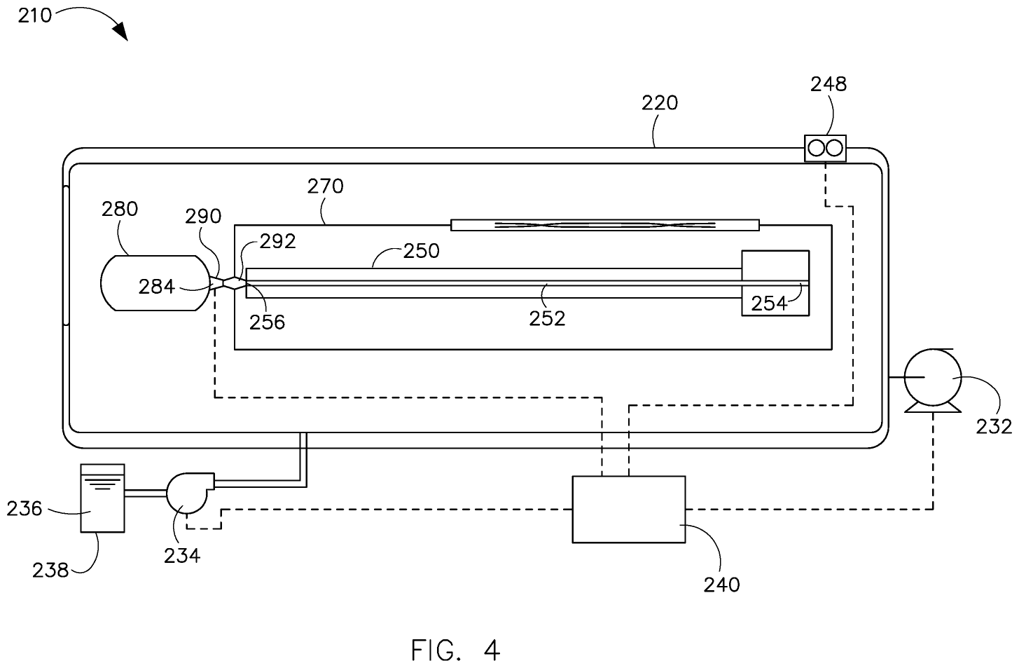

FIG. 4 is a schematic view of a decontamination system 210 that may be used to decontaminate a device 250 having a lumen 252 by connecting a pressure vessel 280 to a second end 256 of the lumen 252. That is, the pressure vessel 280 may be connected to the lumen 252 at the second end 256 rather than at a first end 254. The decontamination system 210 shown in FIG. 4 may operate similar to the decontamination system 10 shown in FIG. 1. As shown in FIG. 4, the decontamination system 210 includes a decontamination chamber 220, a vacuum pump 232, a vaporizer 234, a source of decontaminating substance 236 maintained in a package 238, a controller 240, a vent 248, a device 250 positioned within a container 270, and a pressure vessel 280. An opening 284 of the pressure vessel 280 may be connected to the device 250 at the second end 256 of the lumen 252. In some embodiments, the pressure vessel 280 may be connected to the second end 256 of the lumen 252 by connecting the second end 256 to a port 292 which is connected to the valve 290.

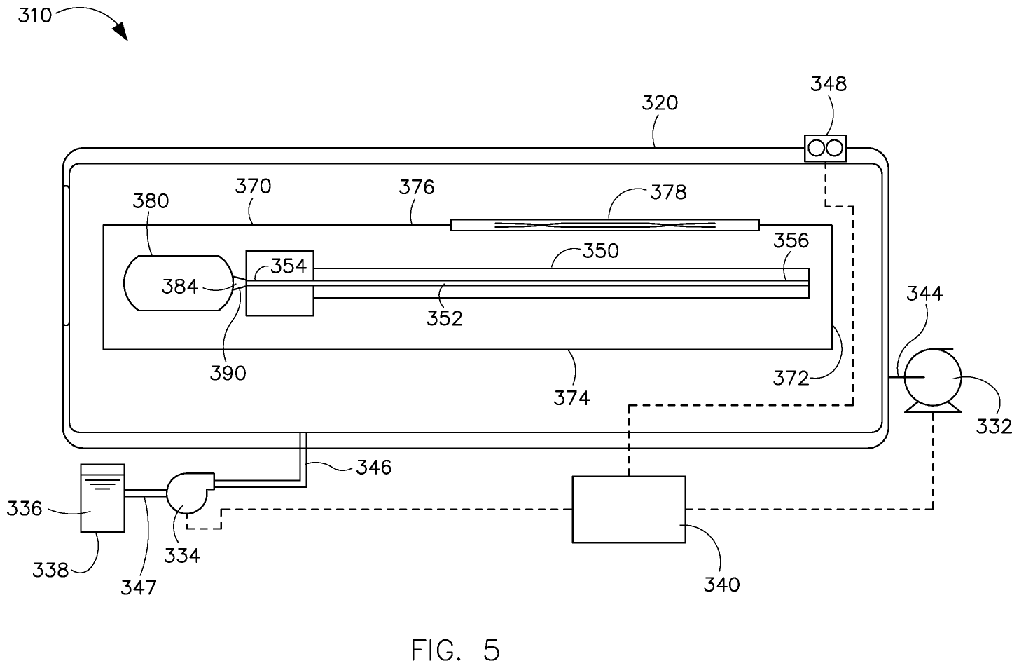

FIG. 5 is a schematic view of decontamination system 310 having a pressure vessel 380 positioned inside a container 370. The decontamination system 310 shown in FIG. 5 may operate similar to the decontamination system 10 shown in FIG. 1. As shown in FIG. 5, the decontamination system 310 includes a decontamination chamber 320, a vacuum pump 332, a vaporizer 334, a source of decontaminating substance 336 maintained in a package 338, a controller 340, a vent 348, and a pressure vessel 380 positioned within a container 370. The vacuum pump 332 is connected to the decontamination chamber 320 by conduit 344. The vaporizer 334 is connected to the decontamination chamber 320 by conduit 346. The decontaminating substance 336 is maintained in package 338 and is connected to vaporizer 334 by conduit 347. The pressure vessel 380 has an opening 384 that is connected to a valve 390. A device 350 may be enclosed in the container 370 and connected to the pressure vessel 380 by connecting the device 350 to the valve 390. The valve 390 may be directly connected to a lumen 352 of the device 350. The lumen 352 may have a first end 354 and a second end 356.

As shown in FIG. 5, the container has sides 372, a top 376, and a bottom 374. The container 370 forms an enclosed space and around the device 350 and the pressure vessel 380. The sides 372, the top 376 and the bottom 374 of the container 370 may be flexible or rigid. For example, the container 370 may be a flexible pouch made entirely or substantially entirely from one or more pliable or flexible materials. In another example, the container 370 may be a case or other enclosure formed from a rigid material. In a further example, the container 370 may have a rigid bottom 374 and sides 372, and may have a flexible top 376 or lid. The container 370 may be disposable, or the container 370 may be reusable. The container 370 may be designed to enclose the device 350 and the pressure vessel 380 during a decontamination process, and maintain the device 350 in a decontaminated condition after the container 370 with the device 350 inside is removed from the decontamination chamber 320.

The container 370 may have a section 378 on the sides 372 or the top 376 through which gases such as air and/or vaporized decontaminating substance 336 may pass. For example, the sides 372 or top 376 may include a section 378 that includes a material that allows air to pass through, but prevents contaminating substances and/or microbes from contacting the device 350. Suitable materials that may be used to form the section 378 include, for example, a nonwoven material such as that sold under the tradename Tyvek®.

As shown in FIG. 5, the pressure vessel 380 may be connected to the lumen 352 by connecting the valve 390 to the first end 354 of the lumen 352. In an alternative embodiment, the pressure vessel 380 may be connected to the second end 356 of the lumen 352. As shown in FIG. 5, the valve 390 is positioned within the container 370 and connected to the pressure vessel 380. It is envisioned that in an alternative embodiment, the pressure vessel 380 may be positioned outside the container 370 and the valve 390 may be positioned within the container 370.

In some embodiments, the valve 390 is battery powered and/or remote operated, which may allow the pressure vessel 380 and valve 390 to be placed anywhere within the container 370 without the need for a connection to a power source or controller 340. For example, the valve 390 may be remote operated from outside the container 370. In some embodiments, the valve 390 may include a sensor (not shown) that detects an air pressure inside the decontamination chamber 320. When a suitable air pressure inside the decontamination chamber 320 is detected, the valve 390 may be programmed to either open or close depending on the desired response. In some embodiments, the valve 390 may be powered by a magnetic coupling (not shown) to a power or control source located outside the decontamination chamber 320 which controls the valve 390 inside the decontamination chamber 320.

In some embodiments, a cycle such as the decontaminating cycle 100 described with reference to FIG. 2 may be used to provide decontaminating substance 336 inside the lumen 352. During a decontaminating cycle, liquid decontaminating substance 336 may be provided to the vaporizer 334 through conduit 347. The vaporizer 334 vaporizes the decontaminating substance 336 and provides vaporized decontaminating substance 336 into the decontamination chamber 320 through conduit 346. Decontaminating substance 336 in the decontamination chamber 320 may contact the sides 372, top 376, and bottom 374 of the container 370. Additionally or alternatively, air containing vaporized decontaminating substance 336 may pass from inside the decontamination chamber 320 through the section 378 into the container 370.

The valve 390 may be controlled to the closed position. The vacuum pump 332 may be controlled to increase the pressure inside the decontamination chamber 320 above the pressure inside the pressure vessel 380. Air containing decontaminating substance 336 may be provided inside the decontamination chamber 320 and pass into the container 370 where it may contact the outside of the device 350. The valve 390 may then be controlled to the open position to allow air containing decontaminating substance 336 to flow into the pressure vessel 380 through the lumen 352. In this manner, the lumen 352 may contact a suitable amount of decontaminating substance 336 to decontaminate the lumen 352.

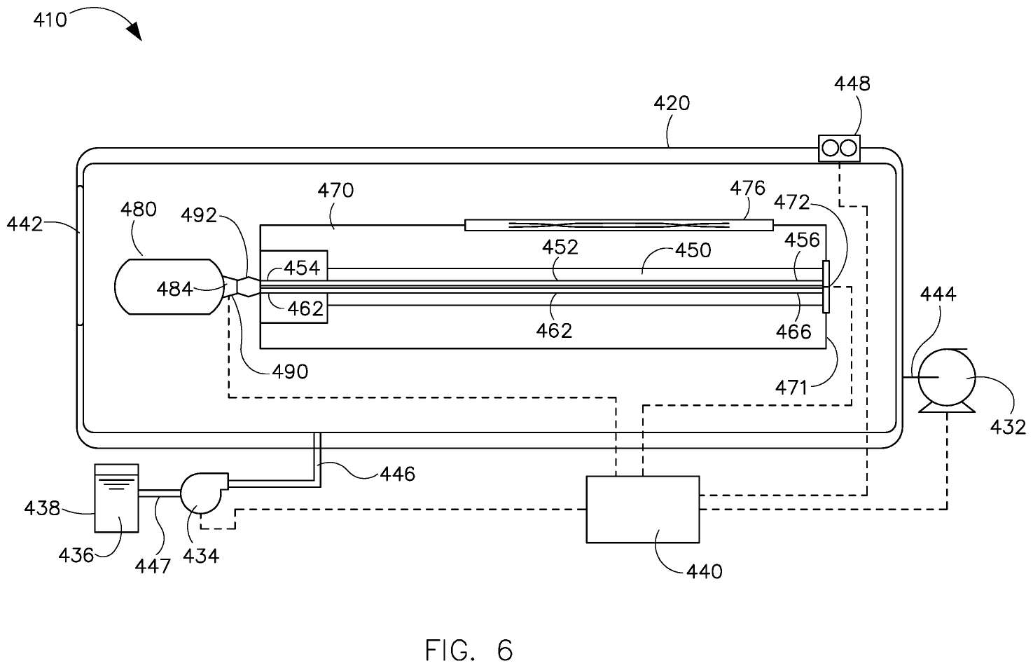

FIG. 6 is a schematic view of a decontamination system 410 configured to decontaminate a device 450 having multiple lumens. The decontamination system 410 shown in FIG. 6 may operate similar to the decontamination system 10 shown in FIG. 1 with similar labeled components in FIG. 1 and FIG. 6 configured to operate substantially the same. As shown in FIG. 6, the decontamination system 410 includes a decontamination chamber 420, a vacuum pump 432, a vaporizer 434, a source of decontaminating substance 436 maintained in a package 438, a controller 440, a door 442, a vent 448, a cover 472, and a pressure vessel 480. The vacuum pump 432 is connected to the decontamination chamber 420 by conduit 444. The vaporizer 434 is connected to the decontamination chamber 420 by conduit 446. The decontaminating substance 436 is maintained in package 438 and is connected to vaporizer 434 by conduit 447. The pressure vessel 480 has an opening 484 that is connected to a valve 490 which is connected to a port 492. The device 450 is enclosed within a container 470 having a section 478 through which gases such as air and/or vaporized decontaminating substance 436 may pass between the container 470 and inside the decontamination chamber 420. The controller 440 is connected to the vacuum pump 432, vaporizer 434, vent 448, cover 472, pressure vessel 480, and/or valve 490, and is configured to control these items.

As shown in FIG. 6, the device 450 includes first lumen 452 and second lumen 462. In still further examples, the device 450 may have more than two lumens. That is, the device 450 may include a third lumen, a fourth lumen, etc. Although the device 450 is shown in FIG. 6 with first lumen 452 and second lumen 462 side by side for illustrative purposes, it is envisioned that the decontamination system 410 may be used to decontaminate devices having first lumen 452 and second lumen 462 arranged in additional or alternative configurations. For example, the device may have first and second lumen 452, 462 arranged coaxially. That is, first lumen 452 may be inside second lumen 462 with both first and second lumens 452, 462 parallel along the length. The first lumen 452 has a first end 454 and a second end 456, and the second lumen has a first end 464 and a second end 466.

As shown in FIG. 6, the port 492 is positioned on the container 470. The port 492 may be directly connected to first and second lumens 452, 462 of the device 450. The port 492 is also connected to the valve 490, and the valve 490 is connected to the pressure vessel 480. In this configuration, the pressure vessel 480 is directly connected to first and second lumen 452, 462 through valve 490 and port 492. The port 492 may be connected to the valve 490 and/or the first and second lumens 452, 464 using any suitable connection such as one or more threaded connections or a quick snap connection. The port 492 may include a material across the cross section of the port 492 that allows gas such as air containing vaporized decontaminating substance 436 to pass through. As shown in FIG. 6, the pressure vessel 480 is connected to the first ends 454, 464 of the first and second lumens 452, 462. In an alternative embodiment, the pressure vessel 480 may be connected to the second ends 456, 466 of the first and second lumens 452, 462.

As shown in FIG. 6, the cover 472 is connected to the second ends 456, 466 of the first and second lumens 452, 462. In some instances, the cover 472 may be connected to the opposite ends of the first and second lumens 452, 462 that are connected to the pressure vessel 480. That is, in an alternative embodiment the second ends 456, 466 of the first and second lumens 452, 462 may be connected to the pressure vessel, and the cover 472 may be connected to the first ends 454, 464 of the first and second lumens 452, 462. The cover 472 is configured to control flow into the first and second lumens 452, 462. That is, the cover 472 is connected to the first and second lumens 452, 462 and controls flow through first and second lumens 452, 462 such that air may be controlled to flow through one or the other or first or second lumens 452, 462 separately, or through both at the same time.

In some embodiments, a cycle such as the decontaminating cycle 100 described with reference to FIG. 2 may be used to provide decontaminating substance 436 inside the first lumen 452. That is, liquid decontaminating substance 436 may be provided to the vaporizer 434 through conduit 447. The vaporizer 434 vaporizes the decontaminating substance 436 and provides vaporized decontaminating substance 436 into the decontamination chamber 420 through conduit 446. Decontaminating substance 436 in the decontamination chamber 420 may contact sides 471, top 476, and bottom 474 of the container 470. Air containing vaporized decontaminating substance 436 may pass from inside the decontamination chamber 420 through the section 478 into the container 470. The valve 490 may be controlled to the closed position. The vacuum pump 432 may be controlled to increase the pressure inside the decontamination chamber 420 above the pressure inside the pressure vessel 480. Air containing decontaminating substance 436 may be provided inside the decontamination chamber 420 and pass into the container 470 where it may contact the outside of the device 450. The valve 490 may then be controlled to the open position to allow air containing decontaminating substance 436 to flow into the pressure vessel 480 through the first and second lumens 452, 462. In this manner, the first lumen 452 may contact a suitable amount of decontaminating substance 436 to decontaminate the first lumen 452.

The cover 472 may be controlled between an open or closed position at various times during a decontamination cycle. In one example, the first lumen 452 may have a larger inner diameter than an inner diameter of the second lumen 462. Because the first lumen 452 has a larger inner diameter than the inner diameter of the second lumen 462, it may be preferred to provide a larger volume of air containing decontaminating substance 436 into the first lumen 452 than the second lumen 462, to ensure adequate decontamination of both the first and second lumens 452, 462. The cover 472 may be used to inhibit air containing decontaminating substance 436 from entering into the second lumen 462 until a suitable volume of air has been provided into the first lumen 452. The cover 472 may be used to delay a flow of air from entering into the second lumen 462 until a suitable time after the valve 490 has been opened before allowing a flow of air into the second lumen 462 such that a suitable flow of air is first provided into the first lumen 452. In some instances, the cover 472 may also be used to control a volume of air into first lumen 452 and second lumen 462 to maintain a turbulent flow of air into one or both of first lumen 452 and second lumen 462.

It is also envisioned that the systems and methods disclosed herein may be used to decontaminate two or more devices having a lumen at the same time. That is the decontamination systems described above may enclose two or more pressure vessels, each of which may be connected to a device having one or more lumens. Still further configurations are also envisioned, such as a decontamination system with the pressure vessel and/or the valve outside the decontamination chamber and connected to the device via a fluid channel such as a tube positioned between the pressure vessel and the device.

It is an object of the present application to provide a flow of air containing decontaminating substance into a device having a lumen. By providing a flow of air containing decontaminating substance into the lumen, a more effective system and process for contacting decontaminating substance along the surface of the lumen is provided than a system or process using only passive diffusion. A pressure vessel having a valve can be used to form a pressure difference between a pressure inside of the pressure vessel and a pressure outside of the pressure vessel that is greater than a pressure difference that can be achieved with a pressure vessel having no valve. The systems and methods described herein provide a decontaminating process that requires less decontaminating substance than a process that does not include a pressure vessel. The decontaminating process also ensures that the entire surface of the lumen comes in contact with decontaminating substance. Flowing air containing decontaminating substance through the lumen forces decontamination substance along the entire length of the lumen. An additional benefit of actively flowing decontaminating substance into a lumen is a shorter cycle time required for adequate decontamination of the entire length of the lumen.

The decontamination system and methods disclosed herein have shown to provide effective decontamination of lumens as short as about 1.0 meters, 1.5 meters, or about 2.0 meters in length, to as long as about 3.0 meters, 3.5 meters, or about 4.0 meters in length, or lengths between any pair of the foregoing values, for example, although additional lengths are contemplated. The decontamination system and methods disclosed herein, have shown to provide effective decontamination of devices having a lumen with an inner diameter of as small at about 0.5 mm, 1 mm, or about 1.6 mm, or as wide as about 2.0 mm, 3.0, about 3.5 mm, or a diameter between any pair of the foregoing values, for example, although additional diameters are contemplated. It has been found that amounts of as low as about 0.9 mL, 1.0 mL or about 1.2 mL, or as large as 1.5 mL, 1.8 mL, or about 2.0 mL, or a volume between any pair of the foregoing values, of decontaminating substance containing about 59 wt. % hydrogen peroxide is successful in decontaminating one or more lumens simultaneously.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

1. A system for decontaminating a device having a lumen, the system comprising:
a pressure vessel defining an enclosed space having a fixed volume, the pressure vessel having an opening;
a valve positioned at the opening of the pressure vessel and controllable between an open first position and a closed second position;
a container configured to receive a device having a lumen, the container defining a wall having a section permeable to vaporized decontaminating substance;
the enclosed space of the pressure vessel configured to be in fluid communication with the lumen of the device and the valve configured to control a flow between the lumen and the enclosed space.

2. The system of claim 1, wherein the fixed volume of the enclosed space is larger than a volume of the lumen.

3. (canceled)

4. (canceled)

5. The system of claim 1, wherein the pressure vessel is positioned outside the container.

6. The system of claim 1, wherein the pressure vessel is positioned within the container.

7. The system of claim 1, further comprising a port through the wall of the container, the port having a layer permeable to vaporized decontaminating substance and connected to the valve.

8. The system of claim 1, wherein the valve is positioned within the container.

9. The system of claim 1, wherein the fixed volume of the enclosed space is sized to produce a turbulent flow of air through the lumen after the valve is opened.

10. The system of claim 1, wherein the valve is controlled between the open position and closed position by a controller.

11. The system of claim 1, wherein the device includes two or more lumens.

12. The system of claim 1, further comprising a cover controllable between an open position and a closed position configured to inhibit air containing decontaminating substance from entering the lumen.

13. A system for decontaminating a device having a lumen, the system comprising:
a decontamination chamber defining a first enclosed space;
a container positioned within the first enclosed space for holding a device;
a source of decontaminating substance;
at least one of a vaporizer or atomizer connected to the source of decontaminating substance and the decontamination chamber;
a pressure vessel having an inlet and defining a second enclosed space, the pressure vessel positioned within the first enclosed space; and
a valve connected to the inlet of the pressure vessel and configured to connect to the lumen to provide fluid communication between the second enclosed space and the lumen.

14. The system of claim 13, wherein the lumen has a first end, a second end, a length extending from the first end to the second end, and a cross sectional area, and wherein the first end of the lumen is configured to connect to the valve and the second end of the lumen is in fluid communication with the first enclosed space.

15. The system of claim 13, wherein a volume of the second enclosed space is larger than a volume of the lumen.

16. The system of claim 13, and further comprising a controller positioned outside the decontamination chamber, the controller in communication with the valve for controlling the valve between an open position and a closed position.

17. The system of claim 13, wherein the pressure vessel is positioned within the first enclosed space and outside the container.

18. The system of claim 13, wherein the pressure vessel is positioned within the first enclosed space and within the container.

19. The system of claim 13, and further comprising a port on the container, the port having a layer permeable to vaporized decontaminating substance and connected to the valve.

20. The system of claim 13, wherein the valve is positioned within the container.

21. The system of claim 13, wherein the valve is positioned within the first enclosed space and outside the container.

22. A method of decontaminating a device having a lumen, the method comprising:
enclosing a device having a lumen and a pressure vessel in a decontamination chamber, an inlet of the pressure vessel is connected to the device by a valve such that the lumen forms at least a portion of a fluid path between the decontamination chamber and the pressure vessel when the valve is in an open position and the decontamination chamber and the pressure vessel are not in fluid communication when the valve is in a closed position;
decreasing the pressure within the decontamination chamber to a first pressure with the valve in the open position;
after decreasing the pressure within the decontamination chamber to the first pressure, providing a vaporized decontaminating substance to and increasing the pressure within the decontamination chamber to a second pressure with the valve in the closed position; and
opening the valve to create a turbulent flow of the vaporized decontaminating substance through the lumen.

23. (canceled)

24. (canceled)
